(19) United States
(12) Patent Application Publication
(10) Pub. No.: US 2009/0093776 A1
(43) Pub. Date: Apr. 9, 2009

(54) 3D SOLID OR HOLLOW SILICON MICRONEEDLE AND MICROKNIFE WITH "-" SHAPE STRUCTURE

(76) Inventors: Ruifeng Yue, Beijing (CN); Yan Wang, Beijing (CN); Litian Liu, Beijing (CN)

Correspondence Address: CONNOLLY BOVE LODGE & HUTZ, LLP, P O BOX 2207, WILMINGTON, DE, 19899, US

(21) Appl. No.: 12/296,672

(22) PCT Filed: Aug. 25, 2006
(86) PCT No.: PCT/CN06/02192

§ 371 Date: Oct. 10, 2008

(30) Foreign Application Priority Data
Apr. 10, 2006 (CN) 200610072807.1

(51) Int. Cl. A61M 5/32; A61B 17/32; B44C 1/22

(52) U.S. Cl. 604/272; 606/167; 216/41

(57) ABSTRACT

The present invention discloses a linear-edged 3D solid or hollow microneedle or microknife. A tip edge of the microneedle or microknife is a linear edge parallel to a group of (111) oriented facets of monocrystalline silicon. The linear edge extends along a straight or curved line and has a narrow width. An opening is formed on one or each side adjoining the linear tip edge, or is formed at the middle of the linear tip edge. The opening is communicated with a channel formed from the bottom surface of the microneedle or microknife, so as to form a through hole from the tip to the bottom of the microneedle or microknife. The triangular channel has six side walls of (111) oriented facets. The microneedle or microknife is used for transdermal drug delivery, body fluid withdrawing or the like. Methods for producing a microneedle or microknife are also disclosed.

### TECHNICAL FIELD

The present invention relates to microsurgical devices and microfabrication processes, especially to linear-edged three-dimensional (3D) solid or hollow silicon microneedles and microknives.

### BACKGROUND ART

Human body skin consists of three structural layers, i.e., stratum corneum, active epidermis and dermis. The outmost layer, the stratum corneum, has a thickness of about 10 to 50 μm and is formed by dense keratinocytes. The epidermis, which is under the stratum corneum, has a thickness of about 50 to 100 μm and is formed by active cells and a few nerve tissues, without any blood vessels. The dermis, which is under the epidermis, is the main component of skin and consists of a large amount of active cells, nerve tissues and blood vessel tissues. Traditional injection needles for hypodermic injection generally have a diameter of about 0.4 to 3.4 mm. In order to quickly deliver a drug into blood vessels, the injection needles should be pierced through the skin and deeply into the tissues under the skin, resulting in significant pain in an injection process which is generally carried out by a skilled professional. Modern research shows that the outmost stratum corneum of skin is the main barrier to drug delivery. When using a microneedle or an array of microneedles to deliver drug, the drug can quickly spread out and penetrate into capillary blood vessels to join in systemic circulation, if the drug is delivered into locations just under the stratum corneum without deeply into the dermis. In this way, the drug delivery locations of microneedles do not reach nerve tissues, and thus patients will not feel pain. Microneedles for drug delivery can be manipulated by non-professional people, the usage of which is flexible and convenient, and the drug delivery process can be stopped at any time. For these reasons, microneedles are likely to be accepted by patients. Moreover, hollow microneedles can be used not only for transdermal drug delivery but also for transdermally withdrawing of a small amount of body fluid.

In the prior art, there are references disclosing structures and producing methods for solid or hollow silicon microneedles, including:
1. S. Henry, D. V. McAllister, M. G. Allen, and M. R. Prausnitz. Microfabricated microneedles, “a novel approach to transdermal drug delivery”, J. Pharmaceut. Sci., 87(8) 922-925, 1998.
2. P. Griss, P. Enoksson, H. K. Tolvanen-Laakso, P. Meriläinen, S. Ollmar, and G. Stemme, “Micromachined electrodes for biopotential measurements”, J. Microelectromech. Syst., 10(1) 10-16, 2001.
3. P. Griss, P. Enoksson, and G. Stemme, “Micromachined barbed spikes for mechanical chip attachment”, Sensors and Actuators A, 95: 94-99, 2002.
4. Patrick Griss and Göran Stemme “Side-Opened Out-of-Plane Microneedles for Microfluidic Transdermal Liquid Transfer”, J. Microelectromech. Syst., 12(3) 296-301, 2003.
5. Han J. G. E. Gardeniers, Regina Luttge, Erwin J. W. Berenschot, Meint J. de Boer, Shuki Y. Yeshurun, Meir Hefetz, Ronny van't Oever, and Albert van den Berg, “Silicon Micromachined Hollow Microneedles for Transdermal Liquid Transport”, J. Microelectromech. Syst., 12(6): 855-862, 2003.
6. E. V. Mukerjee, S. D. Collins, R. R. Isseroff, R. L. Smith, “Microneedle array for transdermal biological fluid extraction and in situ analysis”, Sensors and Actuators A, 114: 267-275, 2004.
7. Boris Stoeber and Dorian Liepmann, “Arrays of Hollow Out-of-Plane Microneedles for Drug Delivery”, J. Microelectromech. Syst., 14(3) 472-479, 2005.
8. N. Roxhed, P. Griss and G. Stemme, “Reliable In-vivo Penetration and Transdermal Injection Using Ultra-sharp Hollow Microneedles”, Proceedings of 13th IEEE International Conference on Solid-State Sensors, Actuators and Microsystems, pp. 213-216, Seoul, Republic of Korea, 2005.

Most silicon microneedles disclosed by these references have cylindrical needle tips like that of traditional sewing needles or slanted needle tips like that of traditional injection needles. The silicon microneedles are made of monocrystalline silicon substrate or specifically of monocrystalline silicon substrate of (100) orientation. The producing method of the silicon microneedles involves a process of silicon isotropic etching or a combination of it with anisotropic etching (including wet etching and/or dry etching). Holes in hollow silicon microneedles are formed by DRIE (Deep Reactive Ion Etching) machines. For a hollow silicon microneedle, a cylindrical or elliptic cylindrical through hole is generally formed inside the silicon microneedle and extends substantially perpendicular to the surface of the silicon substrate. Thus the through hole has a circular or elliptic cross-section near the needle tip of the silicon microneedle. It is known that the DRIE machines are expensive and involve high cost in operation and maintenance. On the other hand, the silicon microneedles are machined in individual pieces, which results in a high cost. In addition, forming through holes through the thickness of a monocrystalline silicon substrate, which is generally hundreds micrometers, is time consuming. These factors result in high manufacture cost of traditional silicon microneedles especially hollow silicon microneedles and prevent them from being widely used.

### SUMMARY OF INVENTION

An object of the present invention is to overcome the disadvantages found in prior art by proposing an improved linear-edged 3D solid or hollow silicon microneedle or microknife. To this end, the present invention provides the following features.
1) The microneedle or microknife comprises a tip having a tip edge, wherein the tip edge is a linear edge parallel to a group of (111) oriented facets of monocrystalline silicon, the linear edge extends in a certain length along a straight line or along a curved line formed on a single plane or a single convexly curved surface, and has a narrow width. The microneedle and the microknife may be categorized according to their application. For example, according to their usage, a microneedle is mainly used for piercing while a microknife may be used for piercing and/or cutting. A hollow microneedle or microknife may be used for fluid delivery or withdraw after piercing or cutting. Further, the microneedle and the microknife may be categorized according to their sizes. For example, the linear edge of a microneedle may have a length of 10 nm to 50 μm and a width of 0 to 50 μm, while the linear edge of a microknife may have a length of 50 μm to 5 mm and a width of 0 to 300 μm.
2) For a hollow silicon microneedle or microknife, an opening is formed on one or each side adjoining the linear tip edge, or is formed at the middle of the linear tip edge; the opening has a shape of triangle, trapezoid, hexagon, similar to triangle, similar to trapezoid or similar to hexagon; the opening is communicated with an inwardly-pointed triangular channel formed from the bottom surface of the silicon microneedle or microknife, so as to form a through hole from the tip to the bottom of the microneedle or microknife; and the triangular channel has six side walls of (111) oriented facets.
3) More broadly, a linear edge of a solid or hollow silicon microneedle or microknife of the present invention may have a length of 10 nm to 5 mm and a width of 0 to 300 μm.
4) The silicon microneedle or microknife may be formed as a single piece Alternatively, a plurality of silicon microneedles or microknives may form an array.
5) The material of the silicon microneedle or microknife is monocrystalline silicon. The concrete shape and size of the silicon microneedle or microknife, including the location of the linear tip edge of the microneedle or microknife (at the middle or one side of the microneedle or microknife), as well as the location, shape (for example the shapes shown in the SEM photographs as triangle, trapezoid, similar to triangle or similar to trapezoid) and size of the opening, are determined by the size of a mask pattern of a photo mask used in a process for producing the microneedle or microknife, the thickness of the monocrystalline silicon substrate and operating conditions adopted when wet etching or dry etching the monocrystalline silicon.

The array may comprise microneedles or microknives arranged on the same silicon substrate with a certain pitch. The microneedles or microknives in an array may be solid or hollow microneedles or microknives, or combinations of them.

The present invention further provides a method for producing a hollow microneedle or microknife, comprising the steps of:
(1) applying a mask film on a clean monocrystalline silicon substrate of (110) orientation, the mask film being able to resist silicon anisotropic wet etching solution;
(2) selectively removing a part of the mask film applied on the silicon substrate, so that a pattern on a photo mask is transferred to the silicon substrate, the pattern on the photo mask having a pair of sides parallel with each other which are arranged to be parallel to a group of (111) oriented facets of the silicon during lithographic exposure;
(3) putting the silicon substrate into an silicon anisotropic wet etching solution to anisotropically etch the silicon substrate, so as to obtain an inwardly-pointed triangular channel which has six side walls formed by silicon facets of (111) orientation;
(4) removing all the remaining parts of the mask film from the silicon substrate, and then applying a second mask film on each side of the silicon substrate, the second mask film being able to resist silicon anisotropic and isotropic wet etching solutions or resist silicon dry etching;
(5) selectively removing a part of the mask film applied on one side of the silicon substrate opposite to that formed with the channel, so that a pattern on a photo mask is transferred to the silicon substrate, the pattern on the photo mask having a pair of sides parallel with each other which, during lithographing, are aligned with the group of (111) oriented silicon facets which correspond to the pair of sides mentioned above in step (2);
(6) isotropically and/or anisotropically wet etching and/or dry etching the patterned side of the silicon substrate obtained in step (5), so as to form a hollow microneedle or microknife; and
(7) removing the second mask film from the silicon substrate.

The present invention further provides a method for producing a hollow microneedle or microknife, comprising the steps of:
(1) applying a mask film on a clean monocrystalline silicon substrate of (110) orientation, the mask film being able to resist silicon anisotropic wet etching solution;
(2) selectively removing a part of the mask film applied on the silicon substrate, so that a pattern on a photo mask is transferred to the silicon substrate, the pattern on the photo mask having a pair of sides parallel with each other which are arranged to be parallel to a group of (111) oriented facets of the silicon during lithographic exposure;
(3) isotropically and/or anisotropically wet etching and/or dry etching the patterned side of the silicon substrate to form a hollow microneedle or microknife; and
(4) removing the second mask film from the silicon substrate.

According to the linear-edged 3D solid or hollow silicon microneedle or microknife, the array of them and the corresponding producing methods, a plurality of silicon substrates can be anisotropically wet etched simultaneously without the need of DRIE etching. The monocrystalline silicon substrates of (110) orientation can be produced in batch with inwardly-directed triangular channels each having six side walls of (111) oriented facets. The methods of the present invention are convenient, reliable, reproducible and time and cost effective, and have a high yield. The hollow silicon microneedles and microknives of the present invention can be used for transdermal drug delivery and withdrawing a small amount of body fluid. Meanwhile, the microknives are also applicable in biological, medical and surgical fields such as microsurgical operations.

### BRIEF DESCRIPTION OF THE DRAWINGS

FIG. 1 is a schematic structural view of a hollow silicon microneedle or microknife with triangular openings formed on opposite sides.

FIG. 2 is a sectional view taken along the line A-A of FIG. 1 showing a microneedle or microknife with double through holes.

FIG. 3 is a sectional view taken along the line A-A of FIG. 1 showing a microneedle or microknife with a single through hole.

FIG. 4a is a sectional view taken along the line B-B of FIG. 1 showing a microneedle or microknife with a straight tip edge.

FIG. 4b is a sectional view similar to FIG. 4a showing a microneedle or microknife with a curved tip edge.

FIG. 5 is a schematic structural view of a hollow silicon microneedle or microknife with a trapezoid opening formed on one side.

FIG. 6 is a sectional view taken along the line A-A of FIG. 5.

FIG. 7a is a sectional view taken along the line B-B of FIG. 5 showing a microneedle or microknife with a straight tip edge.

FIG. 7b is a sectional view similar to FIG. 7a showing a microneedle or microknife with a curved tip edge.

FIG. 8 is a schematic structural view of a hollow silicon microneedle or microknife with a curved tip edge and one or two triangular or trapezoid openings extending up to the middle of the linear-edged tip.

FIG. 9 is a schematic structural view of a hollow silicon microneedle or microknife with a triangular opening formed on its one or two sides and a truncated tip edge.

FIG. 10 is a sectional view taken along the line A-A of FIG. 9.

FIG. 11a is a sectional view taken along the line B-B of FIG. 9 showing a microneedle or microknife with a straight tip edge.

FIG. 11b is a sectional view similar to FIG. 11a showing a microneedle or microknife with a curved tip edge.

FIG. 12 is a schematic structural view of a hollow silicon microneedle or microknife with a trapezoid opening formed on its one or two sides and a truncated tip edge.

FIG. 13 is a sectional view taken along the line A-A of FIG. 12.

FIG. 14a is a sectional view taken along the line B-B of FIG. 12 showing a microneedle or microknife with a straight tip edge.

FIG. 14b is a sectional view similar to FIG. 14a showing a microneedle or microknife with a curved tip edge.

FIG. 15 is a bottom view showing an inwardly-pointed triangular channel formed from the bottom surface of a silicon substrate, the channel having six side walls of (111) oriented facets.

FIG. 16 is a partly cut-away perspective view taken in the direction shown by the line A-A of FIG. 15.

FIG. 17 is a SEM photograph of a hollow silicon microneedle or microknife, which has opposite openings, prepared according to Example 1 of the present invention.

FIG. 18 is a SEM photograph of a hollow silicon microneedle or microknife, which has a single opening, prepared according to Example 1 of the present invention.

FIG. 19 is a SEM photograph of an array of hollow silicon microneedles or microknives, each having opposite openings and double channels (so that the two openings are not communicated with each other), prepared according to Example 1 of the present invention.

FIG. 20 is a SEM photograph of an array of hollow silicon microneedles or microknives, each having opposite openings communicated with each other via a single channel, prepared according to Example 1 of the present invention.

FIG. 21 is a SEM photograph of an array oaf solid silicon microneedles or microknives prepared according to Example 1 of the present invention.

FIG. 22 is a SEM photograph of a hollow silicon microneedle or microknife, which has a single triangular opening, prepared according to Example 2 of the present invention.

FIG. 23 is a SEM photograph of a hollow silicon microneedle or microknife, which has a single trapezoid opening, prepared according to Example 2 of the present invention.

FIG. 24 is a SEM photograph of an array of hollow silicon microneedles or microknives prepared according to Example 2 of the present invention.

FIG. 25 is a SEM photograph of an array of solid silicon microneedles or microknives prepared according to Example 2 of the present invention.

FIG. 26 is a SEM photograph showing a inwardly-pointed triangular channel formed by anisotropic etching a monocrystalline silicon substrate of (110) orientation using an aqueous solution of potassium hydroxide, the channel having six side walls of (111) oriented facets and forming a hexagon opening on the bottom surface of the silicon substrate.

FIG. 27 is a flow chart showing a preparing process according to Example 1 of the present invention.

FIG. 28 is a flow chart showing a preparing process according to Example 2 of the present invention.

FIG. 29 is a flow chart showing a preparing process according to Example 3 of the present invention.

FIG. 30 is a SEM photograph of a hollow silicon microneedle or microknife, which has a single triangular opening, prepared according to Example 3 of the present invention.

FIG. 31 is a SEM photograph of an array of hollow silicon microneedles or microknives, each having a single triangular opening, prepared according to Example 3 of the present invention.

FIG. 32 is a SEM photograph of an array of solid silicon microneedles or microknives prepared according to Example 3 of the present invention.

FIG. 33 is a variant to the microneedle or microknife of FIG. 12.

### DETAILED DESCRIPTION OF THE PREFERRED EMBODIMENTS

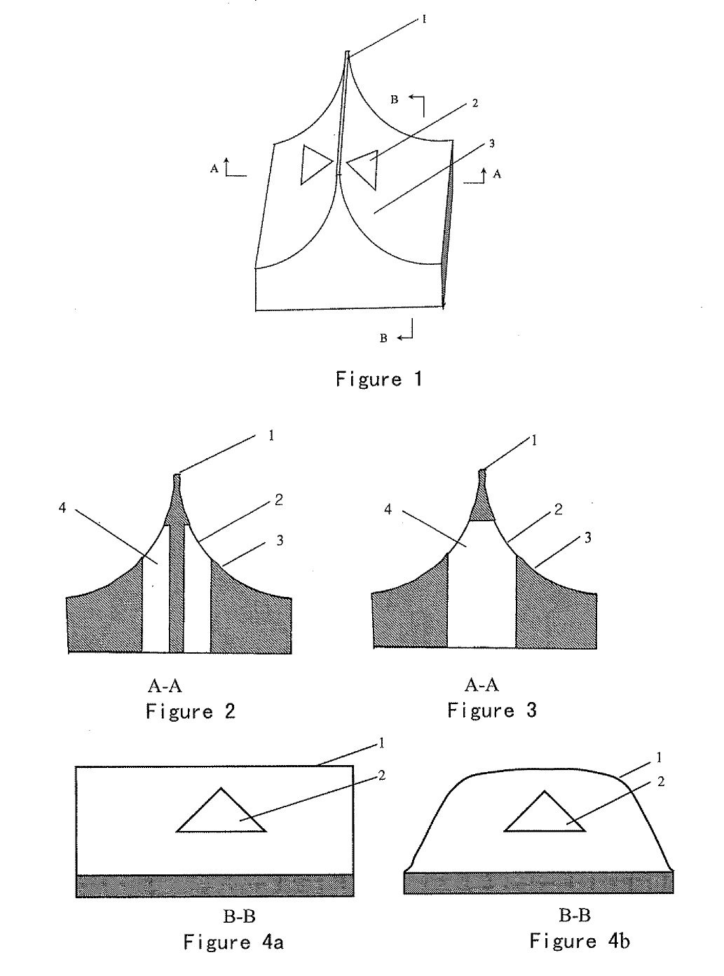

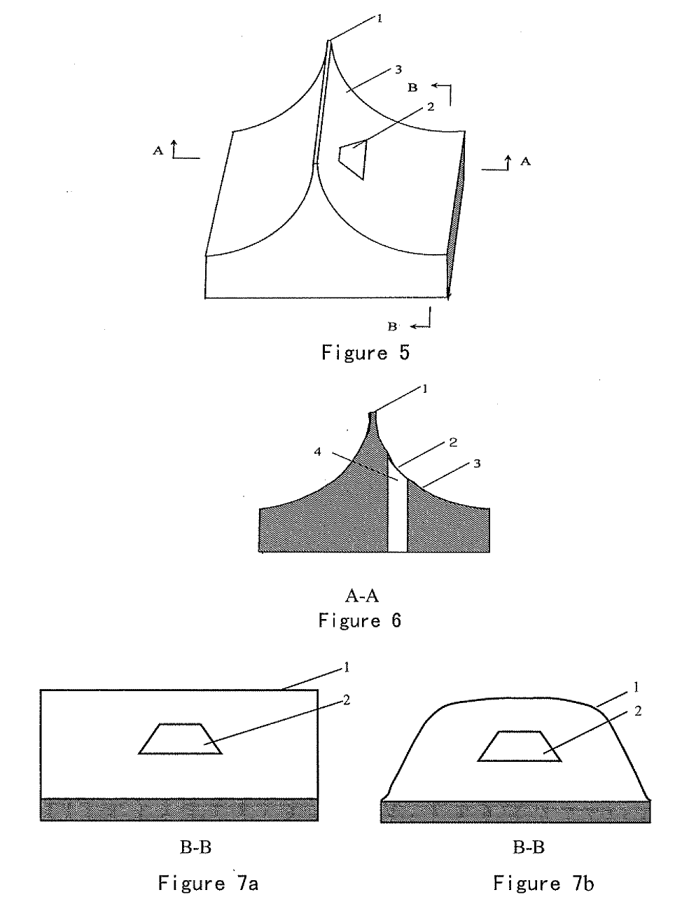

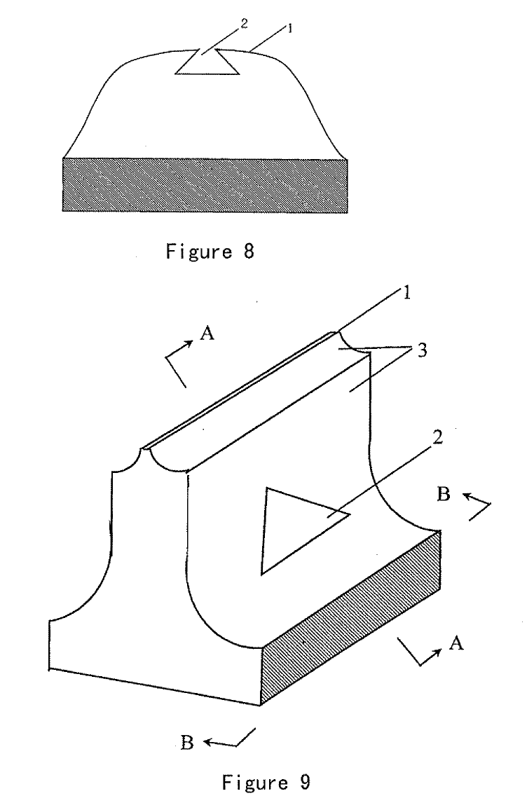

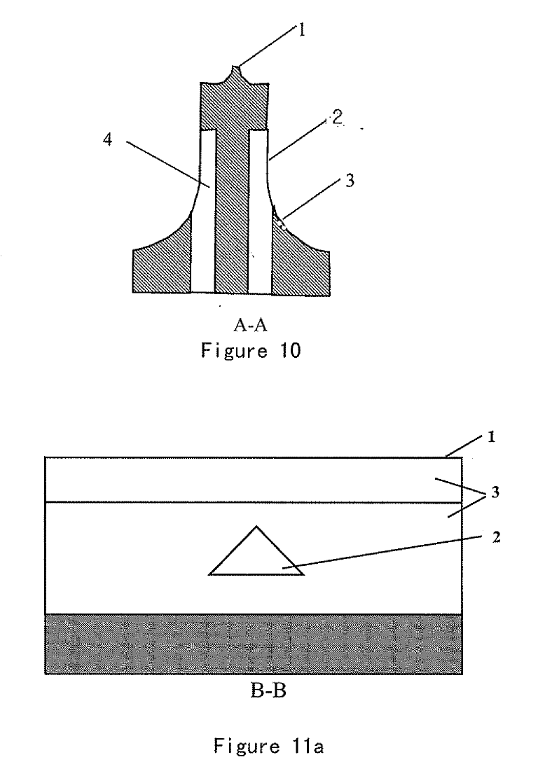

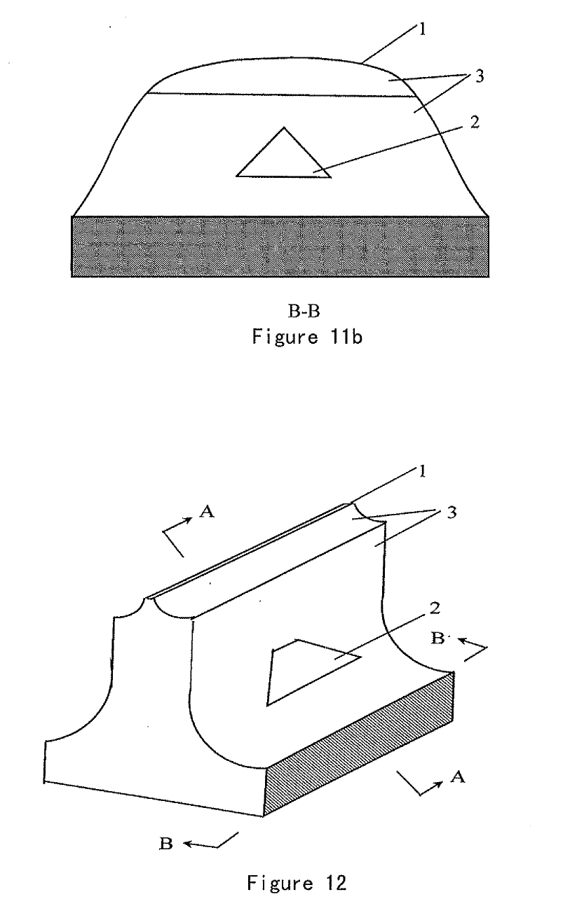

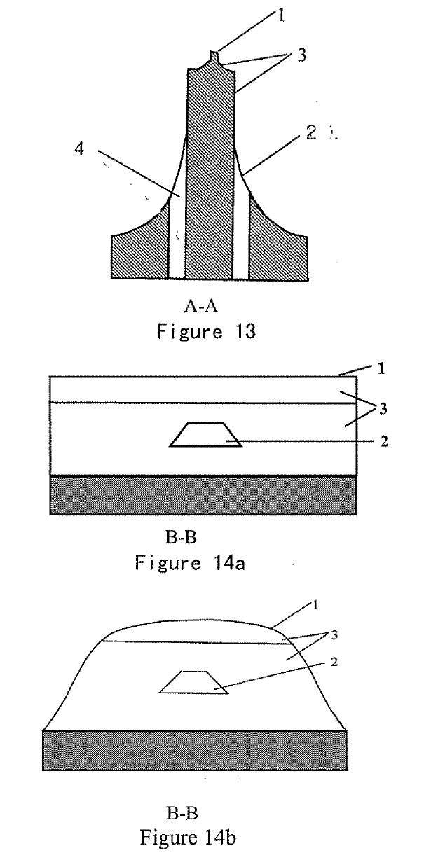

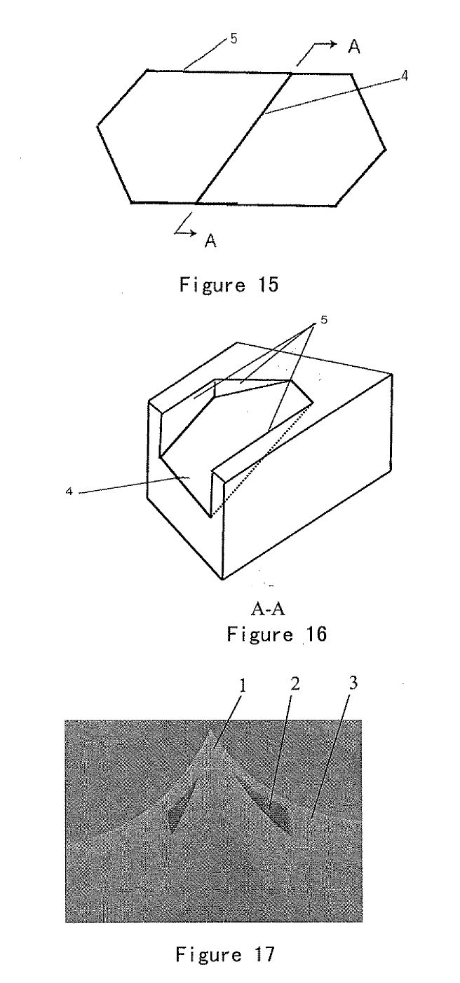

An embodiment of the present invention relates to a linear-edged 3D solid or hollow silicon microneedle or microknife which has structural features as explained below.
1) A tip edge 1 of the tip of the silicon microneedle or microknife is a linear edge parallel to a group of (111) oriented facets 5 (see FIGS. 15, 16 and 26) of monocrystalline silicon. The linear edge extends in a certain length along a straight line or along a curved line formed on a single plane or a single convexly curved surface, and has a narrow width. The microneedle and the microknife may be categorized according to the length of the edge. Some embodiments of the microneedle or microknife are shown in FIGS. 1, 3, 4, 9 and 12.
2) An opening 2 is formed on one or each side 3 adjoining the linear tip edge 1 of the hollow silicon microneedle or microknife, or is formed at the middle of the linear tip edge 1. The opening 2, as shown in FIGS. 1 to 16, may have a shape of triangle, trapezoid, hexagon, similar to triangle, similar to trapezoid or similar to hexagon. The opening is communicated with a triangular channel 4 formed from the bottom surface of the silicon microneedle or microknife, so as to form a through hole from the tip to the bottom of the microneedle or microknife. The triangular channel 4, as shown being inwardly-pointed in FIGS. 15 and 26, has six side walls of (111) oriented facets of silicon.
3) The tip edge of a solid or hollow silicon microneedle or microknife may have a length of 10 nm to 5 mm and a width of 0 to 300 μm.
4) The material of the silicon microneedle or microknife is monocrystalline silicon. The concrete shape and size of the silicon microneedle or microknife, including the location of the linear tip edge of the microneedle or microknife (at the middle or one side of the microneedle or microknife), as well as the location, shape (for example the shapes shown in the SEM photographs as triangle, trapezoid, similar to triangle or similar to trapezoid) and size of the opening, are determined by the size of a mask pattern of a photo mask used in a process for producing the microneedle or microknife, the thickness of the monocrystalline silicon substrate and operating conditions adopted when wet etching or dry etching the monocrystalline silicon.

The silicon microneedle or microknife may be formed as a single piece. Alternatively, a plurality of silicon microneedles or microknives may form an array. The array may comprise microneedles or microknives arranged on the same silicon substrate with a certain pitch. The microneedles or microknives in an array may be solid or hollow microneedles or microknives, or combinations of them. Please refer to FIGS. 20, 21, 24 and 25.

A producing method of a microneedle or microknife having the above features will now be described.
1) Polished opposite sides of a monocrystalline silicon substrate of (110) orientation are each formed with a mask material layer by growth, deposition or coating. The mask material layer may be a film of a single material such as silicon dioxide, silicon nitride, amorphous silicon carbide, other dielectric material or metal, or a composite film of several materials.
2) Then, a photoresist material is coated on the mask material layer formed on a side of the silicon substrate. Then, a mask pattern is obtained from the mask material layer by a pattern transfer process such as lithographing and etching. The mask pattern has a pair of sides parallel with each other which are arranged to be parallel to a group of (111) oriented facets of the silicon during lithographing or etching. Then, the silicon substrate is undergone anisotropic self-stop etching using an silicon anisotropic etching solution to obtain an inwardly-pointed triangular channel 4 which corresponds to the pattern of the mask material layer and has six side walls formed by silicon facets of (111) orientation. The channel forms a hexagon opening on the bottom surface of the silicon substrate, as shown in FIGS. 15, 16 and 26.
3) Then, a photoresist material is provided on the mask material layer formed on another side of the silicon substrate by whirl coating. Then, a mask pattern corresponding to the triangular channel is obtained from the mask material layer by a pattern transfer process such as double-side aligned lithographing and etching. The mask pattern has a pair of sides parallel with each other which are also parallel to the group of (111) oriented facets mentioned in step 2). Then, the silicon substrate is undergone isotropic etching and anisotropic etching using an silicon isotropic etching solution and an anisotropic silicon etching solution respectively or by isotropic dry etching and anisotropic dry etching respectively, so as to form linear tip edges of microneedles or microknives and an array of microneedles or microknives. An opening 2 is formed on one or each the sides 3 adjoining the linear tip edge 1, or is formed at the middle of the linear tip edge 1. The opening 2 is communicated with the triangular channel 4 and has a shape of triangle, trapezoid, similar to triangle or similar to trapezoid.
4) It should be noted that the material for preparing the silicon microneedles or microknives is a monocrystalline silicon substrate of (110) orientation.
5) Finally, the photoresist material and the mask material layer are removed by a wet or dray process.
6) The silicon anisotropic etching solution may be selected from a group of an aqueous solution of potassium hydroxide (with a concentration of 10 to 60 wt %), an aqueous solution of sodium hydroxide (with a concentration of 3 to 50 wt %), EPW (ethylenediamine-pyrocatechol-water with a Mole ratio of 20 to 60% : 0 to 10% : 40 to 80%) and TMAH (an aqueous solution of Tetramethylammonium hydroxide, with a concentration of 5 to 70 wt %).
7) The silicon isotropic etching may be HNA (formed by aqueous solutions of hydrofluoric acid, nitric acid and acetic acid, with a volume ratio of 1 to 30 : 2 to 40 : 5 to 90, wherein the hydrofluoric acid has a concentration of 49% in its aqueous solution, the nitric acid has a concentration of 70% in its aqueous solution, and the acetic acid has a concentration of 99% in its aqueous solution).
8) The dry etching of silicon is performed by a dry etch apparatus (high-pressure plasma etch machine, reactive ion etch machine, inductively coupled plasma etch machine, ion beam milling machine or the like) which isotropically or anisotropically etches silicon using reactive gas or inert gas.
9) In step 3) for etching another side of the silicon substrate to form the tip edges, isotropic and anisotropic wet and/or dry etchings may be performed to the monocrystalline silicon substrate alternatingly. Whether to perform only one of the etchings, and the sequence to perform different etchings, are determined by the concrete structure and size of the silicon microneedles or microknives to be produced.

The producing processes of present invention will now be described in details by means of example and related figures, which will not limit the scope of the structure of the microneedles and microknives as well as their producing method.

### EXAMPLE 1

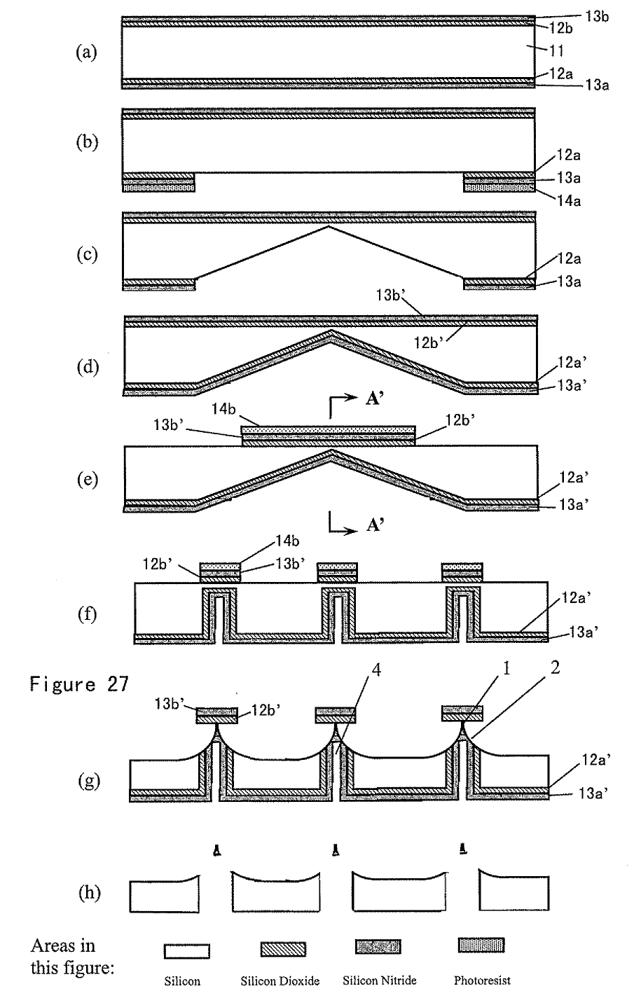

(1) First, general techniques used in micro-electronic field will be adopted. Specifically, a clean monocrystalline silicon substrate 11 of (110) orientation, which has been polished on its opposite sides and has a thickness of 500 μm, is provided. On each of the opposite sides, a silicon dioxide film 12a, 12b having a thickness of 200 nm is grown by thermal oxidization process. Then, on each of the silicon dioxide films 12a, 12b, a silicon nitride film 13a, 13b having a thickness of 200 nm is deposited by an LPCVD (low-pressure chemical vapor deposition) process. Please refer to FIG. 27(a).
(2) Then, a layer of photoresist material 14a having a thickness of about 1 μm is provided on the first side of the silicon substrate by whirl coating. Then, by a pattern transfer process which is generally used in the microelectronic field (including lithographing and etching), a part of the silicon nitride film 13a and a part of the silicon dioxide film 12a on the silicon substrate are selectively removed, so that a pattern on a photo mask is transferred onto the silicon substrate, as shown in FIG. 27(b). The pattern on the photo mask has a pair of sides parallel with each other which are arranged to be parallel to a group of (111) oriented silicon facets during lithographing or etching. Then, the photoresist material 14a is removed by a boiled mixture of sulfuric acid and hydrogen peroxide (with a volume ratio of 3:1). After rinsing, the substrate is put into an aqueous solution of potassium hydroxide having a temperature of 80° C. and a concentration of 30 wt % to anisotropically etch the silicon substrate, so that an upwardly-pointed triangular channel is formed which has six side walls of (111) oriented silicon facets, as shown in FIG. 27(c).
(3) Then, the silicon nitride films 13a, 13b and the silicon dioxide films 12a, 12b are removed in an aqueous solution of hydrofluoric acid having a concentration of 40%. Then, after rinsing, a silicon dioxide film 12a′, 12b′ having a thickness of 200 nm is grown by thermal oxidization process on each of the opposite sides of the substrate. Then, a silicon nitride film 13a′, 13b′ having a thickness of 200 nm is further deposited by an LPCVD process, as shown in FIG. 27(d).
(4) Then, on the second side (the side without the channel) of the silicon substrate, a layer of photoresist material 14b having a thickness of about 1 μm is provided by whirl coating. Then, by a pattern transfer process which is generally used in the micro-electronic field (including lithographing and etching), a part of the silicon nitride film 13b′ and a part of the silicon dioxide film 12b′ on the silicon substrate are selectively removed, so that a pattern on a photo mask is transferred onto the silicon substrate, as shown in FIG. 27(e). The pattern on the photo mask has a pair of sides parallel with each other which, during lithographic exposure using a double-side aligned lithographing machine are aligned with the group of (111) oriented silicon facets which correspond to the pair of sides mentioned above in step (2). FIG. 27(f) shows the cross-section taken along the line A′-A′ of FIG. 27(e).
(5) Then, the photoresist material 14b is removed by a boiled mixture of sulfuric acid and hydrogen peroxide (with a volume ratio of 3:1). After rinsing, the substrate is put into an HNA (formed by hydrofluoric acid, nitric acid and acetic acid with a volume ratio of 3:25:10) solution having a temperature of 50° C. to isotropically etch the silicon substrate. In this process, linear tip edges of microneedles or microknives and consequently an array of microneedles or microknives are formed on the second side of the silicon substrate. An opening is formed on one or each the sides adjoining a linear tip edge, or is formed at the middle of the linear tip edge. The opening is communicated with the triangular channel and has a shape of triangle, trapezoid, similar to triangle or similar to trapezoid, as shown in FIG. 27(g).
(6) Then, the silicon nitride films 13a′, 13b′ and the silicon dioxide films 12a′, 12b′ are removed in an aqueous solution of hydrofluoric acid having a concentration of 40%, and then, after rinsing, the producing process is completed, as shown in FIG. 27(h). The hollow silicon microneedles or microknives made in this way are shown by SEM photographs as explained below.

FIG. 17 shows a SEM photograph of a hollow silicon microneedle or microknife, which has opposite openings, prepared according to Example 1 of the present invention.

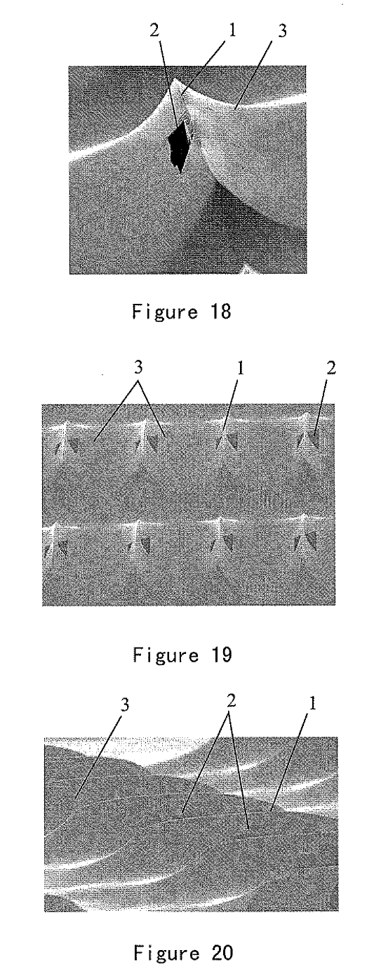

FIG. 18 shows a SEM photograph of a hollow silicon microneedle or microknife, which has a single opening, prepared according to Example 1 of the present invention.

FIG. 19 shows a SEM photograph of an array of hollow silicon microneedles or microknives, each having opposite openings and double channels (so that the two openings are not communicated with each other), prepared according to Example 1 of the present invention.

FIG. 20 shows a SEM photograph of an array of hollow silicon microneedles or microknives, each having opposite openings communicated with each other via a single channel, prepared according to Example 1 of the present invention.

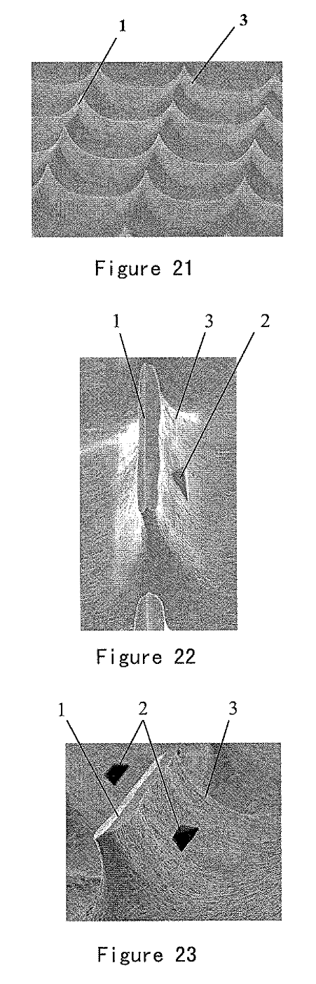

FIG. 21 shows a SEM photograph of an array of solid silicon microneedles or microknives prepared according to Example 1 of the present invention.

### EXAMPLE 2

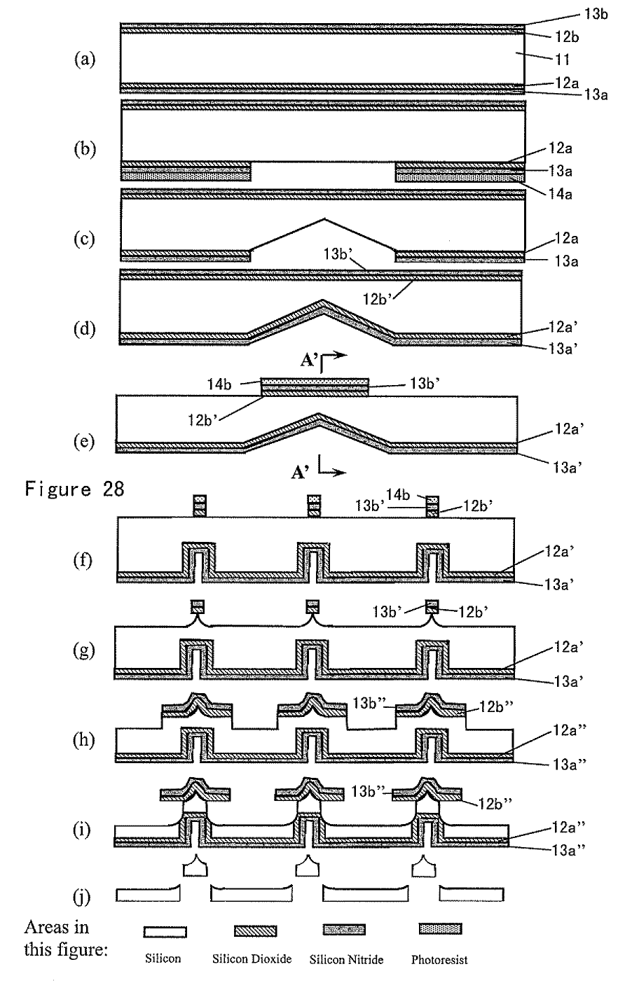

(1) First, general techniques used in micro-electronic field will be adopted. Specifically, a clean monocrystalline silicon substrate 11 of (110) orientation, which has been polished on its opposite sides and has a thickness of 500 μm, is provided. On each of the opposite sides, a silicon dioxide film 12a, 12b having a thickness of 200 nm is grown by thermal oxidization process. Then, on each of the silicon dioxide films 12a, 12b, a silicon nitride film 13a, 13b having a thickness of 200 nm is deposited by an LPCVD process. Please refer to FIG. 28(a).
(2) Then, a layer of photoresist material 14a having a thickness of about 1 μm is provided on the first side of the silicon substrate by whirl coating. Then, by a pattern transfer process which is generally used in the micro-electronic field (including lithographing and etching), a part of the silicon nitride film 13a and a part of the silicon dioxide film 12a on the silicon substrate are selectively removed, so that a pattern on a photo mask is transferred onto the silicon substrate, as shown in FIG. 28(b). The pattern on the photo mask has a pair of sides parallel with each other which are arranged to be parallel to a group of (111) oriented silicon facets during lithographing or etching. Then, the photoresist material 14a is removed by a boiled mixture of sulfuric acid and hydrogen peroxide (with a volume ratio of 3:1). After rinsing, the substrate is put into an aqueous solution of potassium hydroxide having a temperature of 80° C. and a concentration of 30 wt % to anisotropically etch the silicon substrate, so that an upwardly-pointed triangular channel is formed which has six side walls of (111) oriented silicon facets, as shown in FIG. 28(c).
(3) Then, the silicon nitride films 13a, 13b and the silicon dioxide films 12a, 12b are removed in an aqueous solution of hydrofluoric acid having a concentration of 40%. Then, after rinsing, a silicon dioxide film 12a′, 12b′ having a thickness of 200 nm is grown by thermal oxidization process on each of the opposite sides of the substrate. Then, a silicon nitride film 13a′, 13b′ having a thickness of 200 nm is further deposited by an LPCVD process, as shown in FIG. 28(d).
(4) Then, on the second side (the side without the channel) of the silicon substrate, a layer of photoresist material 14b having a thickness of about 1 μm is provided by whirl coating. Then, by a pattern transfer process which is generally used in the micro-electronic field (including lithographing and etching), a part of the silicon nitride film 13b′ and a part of the silicon dioxide film 12b′ on the silicon substrate are selectively removed, so that a pattern on a photo mask is transferred onto the silicon substrate, as shown in FIG. 28(e). The pattern on the photo mask has a pair of sides parallel with each other which, during lithographic exposure using a double-side aligned lithographing machine, are aligned with the group of (111) oriented silicon facets which correspond to the pair of sides mentioned above in step (2). FIG. 28(f) shows the cross-section taken along the line A′-A′ of FIG. 28(e).
(5) Then, the photoresist material 14b is removed by a boiled mixture of sulfuric acid and hydrogen peroxide (with a volume ratio of 3:1). After rinsing, the substrate is put into an HNA (formed by hydrofluoric acid, nitric acid and acetic acid with a volume ratio of 3:25:10) solution having a temperature of 50° C. to isotropically etch the silicon substrate. In this process, linear tip edges having a height of about 10 μm are formed on the second side of the silicon substrate, as shown in FIG. 28(g).
(6) The above step (3) is repeated once: removing the silicon nitride films and the silicon dioxide films, rinsing, and then growing silicon dioxide films 12a″, 12b″ and silicon nitride films 13a″, 13b″ each having a thickness of 200 nm.
(7) Then, on the second side (the side with linear tip edges) of the silicon substrate, a layer of photoresist material (not shown) having a thickness of about 11 μm is provided by whirl coating. Then, by a pattern transfer process which is generally used in the micro-electronic field (including lithographing and etching), a part of the silicon nitride film 13b′ and a part of the silicon dioxide film 12b′ on the silicon substrate are selectively removed, so that a pattern on a photo mask is transferred onto the silicon substrate. The pattern on the photo mask has a pair of sides parallel with each other which, during lithographic exposure, are aligned with the group of (111) oriented silicon facets which correspond to the pair of sides mentioned above in step (2).
(8) Then, the photoresist material 14b is removed by a boiled mixture of sulfuric acid and hydrogen peroxide (with a volume ratio of 3:1). After rinsing, the substrate is put into an aqueous solution of potassium hydroxide having a temperature of 80° C. and a concentration of 30 wt % to anisotropically etch the silicon substrate with an etching depth of about 100 μm, as shown in FIG. 28(h).
(9) Then, the substrate is put into the substrate is put into an HNA (formed by hydrofluoric acid, nitric acid and acetic acid with a volume ratio of 3:25:10) solution having a temperature of 50° C. to isotropically etch the silicon substrate. In this process, linear edged tips having a height of about 200 μm and consequently an array of microneedles or microknives are formed on the second side of the silicon substrate. An opening is formed on one or each the sides adjoining a linear tip edge, or is formed at the middle of the linear tip edge. The opening is communicated with the triangular channel and has a shape of triangle, trapezoid, similar to triangle or similar to trapezoid, as shown in FIG. 28(i).
(10) Then, the silicon nitride films 13a″, 13b″ and the silicon dioxide films 12a″, 12b″ are removed in an aqueous solution of hydrofluoric acid having a concentration of 40%, and then, after rinsing, the producing process is completed, as shown in FIG. 28(j). The hollow silicon microneedles or microknives made in this way are shown by SEM photographs as explained below.

FIG. 22 shows a SEM photograph of a hollow silicon microneedle or microknife, which has a single triangular opening, prepared according to Example 2 of the present invention.

FIG. 23 shows a SEM photograph of a hollow silicon microneedle or microknife, which has a single trapezoid opening, prepared according to Example 2 of the present invention.

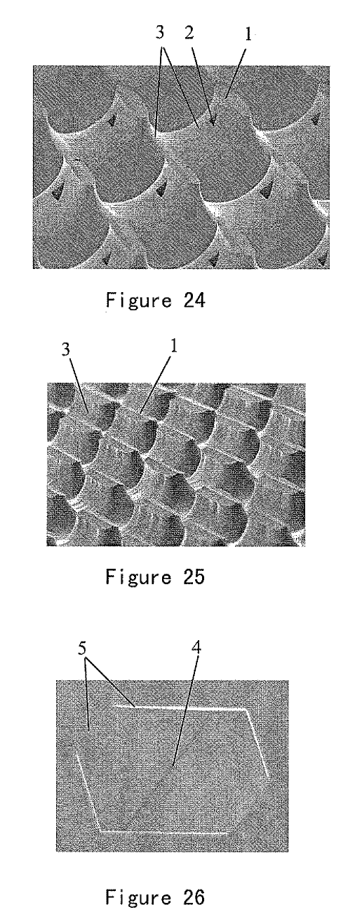

FIG. 24 shows a SEM photograph of an array of hollow silicon microneedles or microknives prepared according to Example 2 of the present invention.

FIG. 25 shows a SEM photograph of an array of solid silicon microneedles or microknives prepared according to Example 2 of the present invention.

FIG. 26 is a SEM photograph showing a inwardly-pointed triangular channel formed by anisotropic etching a monocrystalline silicon substrate of (110) orientation using an aqueous solution of potassium hydroxide, the channel having six side walls of (111) oriented facets and forming a hexagon opening on the bottom surface of the silicon substrate.

### EXAMPLE 3

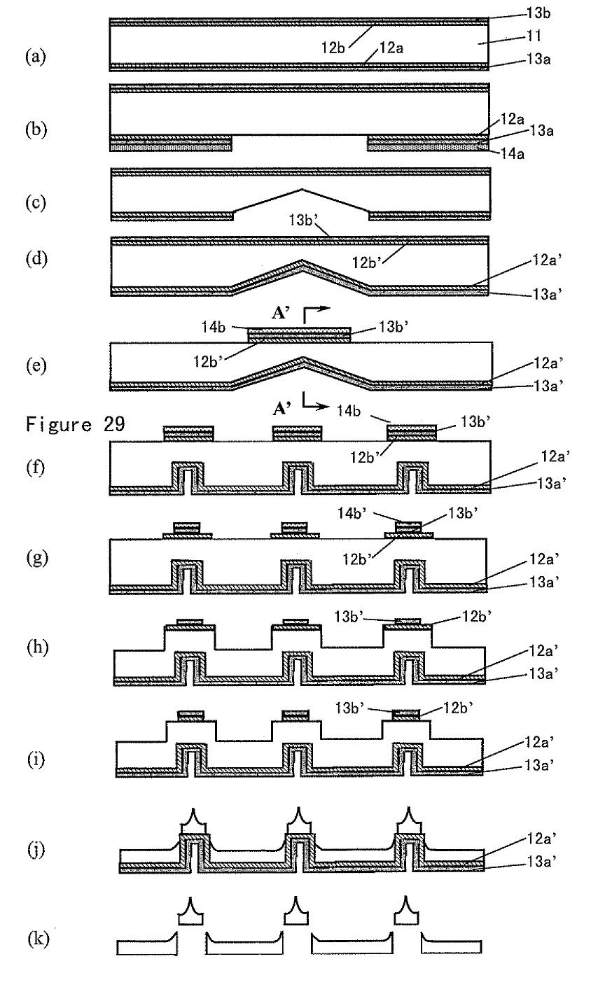

(1) First, general techniques used in micro-electronic field will be adopted. Specifically, a clean monocrystalline silicon substrate 11 of (110) orientation, which has been polished on its opposite sides and has a thickness of 500 μm, is provided. On each of the opposite sides, a silicon dioxide film 12a, 12b having a thickness of 200 nm is grown by thermal oxidization process. Then, on each of the silicon dioxide films 12a, 12b, a silicon nitride film 13a, 13b having a thickness of 200 nm is deposited by an LPCVD process. Please refer to FIG. 29(a).
(2) Then, a layer of photoresist material 14a having a thickness of about 1 μm is provided on the first side of the silicon substrate by whirl coating. Then, by a pattern transfer process which is generally used in the micro-electronic field (including lithographing and etching), a part of the silicon nitride film 13a and a part of the silicon dioxide film 12a on the silicon substrate are selectively removed, so that a pattern on a photo mask is transferred onto the silicon substrate, as shown in FIG. 29(b). The pattern on the photo mask has a pair of sides parallel with each other which are arranged to be parallel to a group of (111) oriented silicon facets during lithographing or etching. Then, the photoresist material 14a is removed by a boiled mixture of sulfuric acid and hydrogen peroxide (with a volume ratio of 3:1). After rinsing, the substrate is put into an aqueous solution of potassium hydroxide having a temperature of 80° C. and a concentration of 30 wt % to anisotropically etch the silicon substrate, so that an upwardly-pointed triangular channel is formed which has six side walls of (111) oriented silicon facets, as shown in FIG. 29(c).
(3) Then, the silicon nitride films 13a, 13b and the silicon dioxide films 12a, 12b are removed in an aqueous solution of hydrofluoric acid having a concentration of 40%. Then, after rinsing, a silicon dioxide film 12a′, 12b′ having a thickness of 200 nm is grown by thermal oxidization process on each of the opposite sides of the substrate. Then, a silicon nitride film 13a′, 13b′ having a thickness of 200 nm is further deposited by an LPCVD process, as shown in FIG. 29(d).
(4) Then, on the second side (the side without the channel) of the silicon substrate, a layer of photoresist material 14b having a thickness of about 1 μm is provided by whirl coating. Then, by a pattern transfer process which is generally used in the micro-electronic field (including lithographing and etching), a part of the silicon nitride film 13b′ and a part of the silicon dioxide film 12b′ on the silicon substrate are selectively removed, so that a pattern on a photo mask is transferred onto the silicon substrate, as shown in FIG. 29(e). The pattern on the photo mask has a pair of sides parallel with each other which, during lithographic exposure using a double-side aligned lithographing machine, are aligned with the group of (111) oriented silicon facets which correspond to the pair of sides mentioned above in step (2). FIG. 29(f) shows the cross-section taken along the line A′-A′ of FIG. 29(e).
(5) Then, the photoresist material 14b is removed by a boiled mixture of sulfuric acid and hydrogen peroxide (with a volume ratio of 3:1). After rinsing, the patterned surface of the silicon substrate formed in step (4) is further provided with a layer of photoresist material 14b′ having a thickness of about 1 μm is provided by whirl coating. Then, by a pattern transfer process which is generally used in the micro-electronic field (including lithographing and etching), a part of the silicon nitride film 13b′ is selectively removed, so that a pattern on a photo mask is transferred onto the silicon substrate, as shown in FIG. 29(g).
(6) Then, the photoresist material 14b′ is removed by a boiled mixture of sulfuric acid and hydrogen peroxide (with a volume ratio of 3:1). After rinsing, the substrate is put into an aqueous solution of potassium hydroxide having a temperature of 80° C. and a concentration of 30 wt % to anisotropically etch the silicon substrate with an etching depth of about 150 μm, as shown in FIG. 29(h).
(7) Then, the exposed silicon dioxide film 12b′ on the silicon substrate is removed by a buffer solution of hydrofluoric acid, as shown in FIG. 29(i). Then, the substrate is put into the substrate is put into an HNA (formed by hydrofluoric acid, nitric acid and acetic acid with a volume ratio of 3:25:10) solution having a temperature of 50° C. to isotropically etch the silicon substrate.

In this process, linear edged tips having a height of about 200 μm and consequently an array of microneedles or microknives are formed on the second side of the silicon substrate. An opening is formed on one or each the sides adjoining a linear tip edge, or is formed at the middle of the linear tip edge. The opening is communicated with the triangular channel and has a shape of triangle, trapezoid, similar to triangle or similar to trapezoid, as shown in FIG. 29(j).
(8) Then, the silicon nitride films 13a′, 13b′ and the silicon dioxide films 12a′, 12b′ are removed in an aqueous solution of hydrofluoric acid having a concentration of 40%, and then, after rinsing, the producing process is completed, as shown in FIG. 29k). The hollow silicon microneedles or microknives made in this way are shown by SEM photographs as explained below.

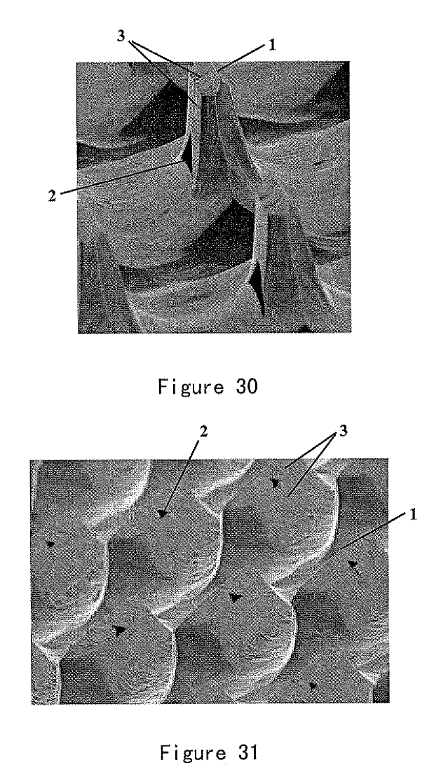

FIG. 30 shows a SEM photograph of a hollow silicon microneedle or microknife, which has a single triangular opening, prepared according to Example 3 of the present invention.

FIG. 31 shows a SEM photograph of an array of hollow silicon microneedles or microknives, each having a single triangular opening, prepared according to Example 3 of the present invention.

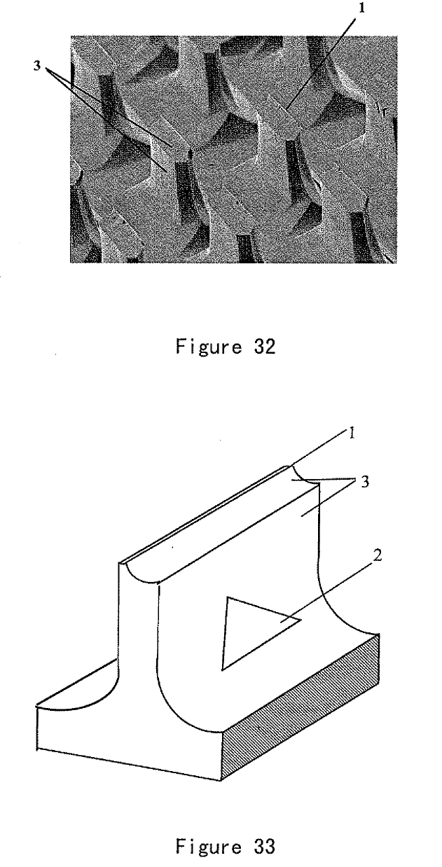

FIG. 32 shows a SEM photograph of an array of solid silicon microneedles or microknives prepared according to Example 3 of the present invention.

1. A 3D silicon microneedle comprising a tip having a tip edge, wherein the tip edge is a linear edge parallel to a group of (111) oriented facets of monocrystalline silicon, the linear edge extends in a certain length along a straight line or along a curved line formed on a single plane or a single convexly curved surface, and has a narrow width, and the linear edge has a length of 10 nm to 50 μm and a width of 0 to 50 μm.

2. The silicon microneedle according to claim 1, wherein the silicon microneedle is solid or hollow.

3. The silicon microneedle according to claim 1, wherein, for a hollow silicon microneedle, an opening is formed on one or each side adjoining the linear tip edge, or is formed at the middle of the linear tip edge, the opening has a shape of triangle, trapezoid, hexagon, similar to triangle, similar to trapezoid or similar to hexagon, the opening is communicated with an inwardly-pointed triangular channel formed from the bottom surface of the silicon microneedle, so as to form a through hole from the tip to the bottom of the microneedle, and the triangular channel has six side walls of (111) oriented facets.

4. The silicon microneedle according to claim 1, wherein the silicon microneedle is provided as a single piece or an array of a plurality of silicon microneedles.

5. The silicon microneedle according to claim 1, wherein the material of the silicon microneedle is monocrystalline silicon; and the concrete shape and size of the silicon microneedle, including the location of the linear tip edge of the microneedle, which is selected from the middle or one side of the microneedle, as well as the location, shape and size of the opening, are determined by the size of a mask pattern of a photo mask used in a process for producing the microneedle, the thickness of the monocrystalline silicon substrate and operating conditions adopted when wet etching or dry etching the monocrystalline silicon.

6. The silicon microneedle according to claim 4, wherein the array comprises microneedles arranged on the same silicon substrate with a certain pitch, and the microneedles in an array comprise solid or hollow microneedles, or combinations of them.

7. A 3D silicon microknife comprising a tip having a tip edge, wherein the tip edge is a linear edge parallel to a group of (111) oriented facets of monocrystalline silicon, the linear edge extends in a certain length along a straight line or along a curved line formed on a single plane or a single convexly curved surface, and has a narrow width, and the linear edge has a length of 50 μm to 5 mm and a width of 0 to 300 μm.

8. The silicon microknife according to claim 7, wherein the silicon microknife is solid or hollow.

9. The silicon microknife according to claim 7, wherein, for a hollow silicon microknife, an opening is formed on one or each side adjoining the linear tip edge, or is formed at the middle of the linear tip edge, the opening has a shape of triangle, trapezoid, hexagon, similar to triangle, similar to trapezoid or similar to hexagon, the opening is communicated with an inwardly-pointed triangular channel formed from the bottom surface of the silicon microknife, so as to form a through hole from the tip to the bottom of the microknife, and the triangular channel has six side walls of (111) oriented facets.

10. The silicon microknife according to claim 7, wherein the silicon microknife is provided as a single piece or an array of a plurality of silicon microknives.

11. The silicon microknife according to claim 7, wherein the material of the silicon microknife is monocrystalline silicon; and the concrete shape and size of the silicon microknife, including the location of the linear tip edge of the microknife, which is selected from the middle or one side of the microknife, as well as the location, shape and size of the opening, are determined by the size of a mask pattern of a photo mask used in a process for producing the microknife, the thickness of the monocrystalline silicon substrate and operating conditions adopted when wet etching or dry etching the monocrystalline silicon.

12. The silicon microknife according to claim 10, wherein the array comprises microknives arranged on the same silicon substrate with a certain pitch, and the microknives in an array comprise solid or hollow microknives, or combinations of them.

13. A method for producing a hollow microneedle or microknife, comprising the steps of:
(1) applying a mask film on a clean monocrystalline silicon substrate of (110) orientation, the mask film being able to resist silicon anisotropic wet etching solution;
(2) selectively removing a part of the mask film applied on the silicon substrate, so that a pattern on a photo mask is transferred to the silicon substrate, the pattern on the photo mask having a pair of sides parallel with each other which are arranged to be parallel to a group of (111) oriented facets of the silicon during lithographic exposure;
(3) putting the silicon substrate into an silicon anisotropic wet etching solution to anisotropically etch the silicon substrate, to obtain an inwardly-pointed triangular channel which has six side walls formed by silicon facets of (111) orientation;
(4) removing all the remaining parts of the mask film from the silicon substrate, and then applying a second mask film on each side of the silicon substrate, the second mask film being able to resist silicon anisotropic and isotropic wet etching solutions or resist silicon dry etching;
(5) selectively removing a part of the mask film applied on one side of the silicon substrate opposite to that formed with the channel, so that a pattern on a photo mask is transferred to the silicon substrate, the pattern on the photo mask having a pair of sides parallel with each other which, during lithographing, are aligned with the group of (111) oriented silicon facets which correspond to the pair of sides mentioned above in step (2);
(6) isotropically and/or anisotropically wet etching and/or dry etching the patterned side of the silicon substrate obtained in step (5), so as to form a hollow microneedle or microknife; and
(7) removing the second mask film from the silicon substrate.

14. The producing method according to claim 13, further comprising the following step between step (5) and step (6):
selectively removing a part of the mask film applied on the patterned side of the silicon substrate obtained in step (5), so that a pattern on another photo mask is transferred to the silicon substrate

15. The producing method according to claim 13, wherein the mask film applied in step (1) and/or step (4) is a silicon dioxide film or a silicon nitride film or a complex film of silicon dioxide and silicon nitride.

16. The producing method according to claim 13, wherein the silicon anisotropic wet etching solution is selected from a group of: an aqueous solution of potassium hydroxide, an aqueous solution of sodium hydroxide, EPW, and TMAH; and
the silicon isotropic wet etching is HNA.

17. A method for producing a hollow microneedle or microknife, comprising the steps of:
(1) applying a mask film on a clean monocrystalline silicon substrate of (110) orientation, the mask film being able to resist silicon anisotropic wet etching solution;
(2) selectively removing a part of the mask film applied on the silicon substrate, so that a pattern on a photo mask is transferred to the silicon substrates the pattern on the photo mask having a pair of sides parallel with each other which are arranged to be parallel to a group of (111) oriented facets of the silicon during lithographic exposure;
(3) isotropically and/or anisotropically wet etching and/or dry etching the patterned side of the silicon substrate to form a hollow microneedle or microknife; and
(4) removing the second mask film from the silicon substrate.

18. The producing method according to claim 17, further comprising the following step between step (2) and step (3) or in step (3):
selectively removing a part of the mask film applied on the patterned side of the silicon substrate, so that a pattern on another photo mask is transferred to the silicon substrate.

19. The producing method according to claim 17, wherein the mask film applied in step (1) is a silicon dioxide film or a silicon nitride film or a complex film of silicon dioxide and silicon nitride.

20. The producing method according to claim 17, wherein the silicon anisotropic wet etching solution is selected from a group of: an aqueous solution of potassium hydroxide, an aqueous solution of sodium hydroxide, EPW, and TMAH; and
the silicon isotropic wet etching is HNA.
